(19) United States
(12) Patent Application Publication
(10) Pub. No.: US 2022/0218253 A1
(43) Pub. Date: Jul. 14, 2022

(54) Impairment Detection Method and Devices

(71) Applicant: Don P. SEIDENSPINNER, LAS VEGAS, NV (US)

(72) Inventor: Don P. SEIDENSPINNER, LAS VEGAS, NV (US)

(21) Appl. No.: 17/147,185

(22) Filed: Jan. 12, 2021

(51) Int. Cl. A61B 5/16; G06K 9/00; A61B 5/18

(52) U.S. Cl. CPC A61B 5/163; G06K 9/00604; A61B 5/18; G06K 9/00718; G06K 9/0061

(57) ABSTRACT

The present invention relates to detecting impairment. Impairment is generally systemic and may impact all regions of the body, including muscle responses and cognitive responses. An inverse relationship typically exists between involuntary muscle reaction time to a stimulus and level of impairment. Reaction time is impacted by many intoxicants, as well as by one or more medical conditions. In preferred form, a video records an involuntary response to a stimulus such as a flash of light, a loud sound, touch or an electrical stimulation. Software is utilized to deliver the stimulus, acquire, analyze, and output the reaction time and/or magnitude of the subject. Classification of the degree of impairment is then determined, e.g., using of comparative datasets and/or algorithms.

### CROSS REFERENCE

This application claims the benefit of U.S. Provisional Application No. 62/960,508 filed 13 Jan. 2020.

### FIELD OF THE INVENTION

This disclosure generally relates to a method, system employing the method, and devices for enabling the method that assesses impairment, more specifically, by assessing involuntary reaction latency and magnitude of at least one involuntary muscle in response before, during and after application of an external stimulus. The device for this impairment evaluation may be a handheld unit, e.g., a smart phone, suitably programmed to provide the delivery of a stimulus, a record of the response to the stimulus, and perform at least a portion of the analysis of the response for determination of possible impairment of the measured individual.

### BACKGROUND

Need for assessing possible impairment of an individual's ability to operate a motor vehicle, boat, machinery, or conduct other possibly injurious tasks is a widespread need in everyday life. For example, impaired ability to operate a motor vehicle may result in property destruction, injury, or death.

Accordingly, a variety of approaches have been developed to detect such impairment particularly in the case of driving under the influence of alcohol. Primary among these tests is the assessment of an individual's blood alcohol level, either through direct measurement of blood alcohol content or indirectly through measurement of alcohol or its chemical by-products through exhalation or in sweat.

As these methods are intrusive, more rapid, less intrusive screening techniques are employed to first ascertain if an individual exhibits signs of impaired function and then, if so, resorting to more definitive tests for possible agents, e.g., drugs or alcohol, causing this impairment, may be employed.

A familiar form of screening for impairment is the Standardized Field Sobriety Test (SFST) endorsed by the National Highway Traffic and Safety Administration (NHTSA). This test consists of the horizontal gaze nystagmus (HGN), walk-and-turn (WAT) and one-leg stand (OLS) tests. As the scoring for these tests relies on the observational skills of the examiner, the subjective nature of this process may lead to discordant findings.

In order to provide a more quantitative assessment of impairment, a variety of monitoring devices have been proposed. Several of these approaches involve the placement of an eye movement monitoring device in contact with the individual's face to allow more quantitative tracking of eye movement (as part of the SFST). However, such devices are cumbersome and have not proved a substantive improvement over the basic SF ST.

Accordingly, there remains a need for a rapid, easy-to-use approach for assessing impairment, whether due to use of prescribed drugs, illicit drugs, alcohol, marijuana, medical conditions, or from other causes, and that may be employed by individuals, groups or organizations to ensure health and safety in various scenarios and use cases.

### PRIOR ART

### U.S. Patent & Patent Applications

U.S. Pat. No. 4,057,724
U.S. Pat. No. 8,783,870
U.S. Pat. No. 8,317,328
U.S. Pat. No. 8,783,869
U.S. Pat. No. 9,380,958
U.S. Pat. No. 9,481,245
U.S. Pat. No. 8,319,637
U.S. Pat. No. 4,912,458
U.S. Pat. No. 4,901,058
U.S. Pat. No. 4,723,625
U.S. Pat. No. 4,738,333
U.S. Pat. No. 4,697,666
U.S. Pat. No. 4,902,628
U.S. Pat. No. 6,167,746
U.S. Pat. No. 9,228,997
U.S. Pat. No. 9,980,642
U.S. Pat. No. 8,374,701
U.S. Pat. No. 9,171,131
U.S. Pat. No. 10,386,358
20180140250
20150251660
20100328066
20140002798
20140327879
20180313818
20170307588
20160161468
20140311215
20170128005
20190298257
20170350877
20180128810
20140320285
20200175686
20180153399
20170337441

### Other Publications

J. Jason McAnany, et al. “iPhone-based Pupillometry: A Novel Approach for Assessing the Pupillary Light Reflex”, Optom Vis Sci 2018; Vol 95(10)
O. Bergamin and R. H. Kardon “Latency of the Pupil Light Reflex: Sample Rate, Stimulus Intensity, and Variation in Normal Subjects” Investigative Ophthalmology & Visual Science, April 2003, Vol. 44, No. 4
D. Dhingra, S. Kaur, and J. Ram “Illicit drugs: Effects on eye” Indian J Med Res. 2019 September; 150(3): 228-238.
C. A. Hall and R. P. Chilcott “Eyeing up the Future of the Pupillary Light Reflex in Neurodiagnostics” Diagnostics 2018, 8, 19

### SUMMARY OF THE INVENTION

Disclosed herein is a novel method and system for assessing impairment. In brief, the method consists of provoking the subject with at least one stimulus while monitoring before, during and/or after the response to the stimulation of at least one muscle exhibiting an autonomic response to the stimulus. In preferred instances, one or more of the facial muscles, specifically those of the eye iris, are monitored. Monitoring of muscles may include, but not limited to, muscle movement, position or shape, e.g., pupil diameter, to ascertain variance in the response such as a delay and/or magnitude of the response from normative values.

Assessment of impairment likelihood and degree of impairment may then be determined in a quantitative fashion. Quantitative results may then be obtained with use of one or more analysis techniques, such as machine learning algorithms, naive Bayes classifier, neural networks, etc. Monitoring subjects may then be presented with the results and the results may be stored for future reference along with the monitoring data.

In one embodiment, the method relies on the sequential application of an audible stimulus such as a brief loud noise, verbal instructions or questions using a device such as a smart phone which then continues to record the subject before, during and after the stimulus is presented to the subject under test. Should an involuntary muscle response to the stimulus not be observed, then the method may be repeated.

In alternate embodiments, alternative forms of stimulation maybe employed, e.g., a visible light flash, a mild electrical shock, or physical contact, to elicit a response from the subject.

Data from the recording is then analyzed for concordance with the timing and magnitude of the response with one or more training datasets or values representative of normative and impaired responses. Determination of impairment severity may then be presented to screening individual for subsequent action.

In one embodiment, the delivery of the stimulus, the recorder of the stimulus and response, and the display of the results are enabled within a single device. A form of this device is a computational device capable of rendering a webpage, has a video camera and other functionalities such as a “smart” phone having sufficient capabilities such as flash (stimulus) functionality, adequate video frame recording rates, focusing, and resolution plus processing power and memory to accomplish these functions.

In alternate embodiments, the delivery of the stimulus may be through a separate device, an attached component, or external action from an individual. In such instances, synchronization of the timing of stimulus delivery and recording of the subject's response is required should two or more devices be involved. For example, a flash of light as a stimulus may be delivered by a flash unit that is not affixed to a video recording device but whose activity is coordinated, e.g., through use of control logic software and wired or wireless communication as part of an overall system, to synchronize activities between delivery of the stimulus and recording of the physical reaction to the stimulus.

In system-based embodiments, the device providing stimulus and possible recording one or more responses to the stimulus is separate from one or more devices or computational systems performing the analysis that determines various aspects of the response, e.g., onset timing and magnitude of response, as well as likelihood of impairment as well as any subsequent action(s) to be taken. Communication between devices and computational systems may occur using the internet and wired or wireless means for transference of programs, data, or instructions.

In still other embodiments of the invention, two or more body sites are measured in response to the delivery of one or more stimuli and are analyzed together to determine likelihood of impairment. As an example, comparison of two eyes simultaneously stimulated and measured may be made to determine possible selective neurological damage to a subject's nervous system, optical nerve, or brain regions as compared to a more generalized impairment of neurological function.

Additional embodiments and variations of the methods, systems, and devices for accomplishing the present invention are readily conceivable and therefore the scope of the present invention is not constrained to the embodiments presented above. It is understood that the use of the word “and” within the disclosure is understood to mean “and/or” where one or more conditions or structures are conceivably employed.

### BRIEF DESCRIPTION OF THE DRAWINGS

FIG. 1—Diagram of one embodiment of a system employing the method of the invention

FIG. 2—Diagram illustrating operational software flow in a device employing the method of the invention

FIG. 3—Graph of pupillary light reflex response and measurable elements of the response

FIG. 4—Illustration of hypothetical graph of data utilized for analysis in one embodiment of the invention

FIG. 5—Diagram of the system of the invention utilized with two different devices

### DEFINITIONS

In the context of the present invention, the following words or terms are intended to mean:

“impairment”—an alteration in onset, latency, acceleration, magnitude, onset, or duration from normal values representative of that individual or population of one or more involuntary motor reflexes in response to a stimulus or compared with a previous measurement of the subject. As this form of impairment is often due to a reduction in nerve signal transmission rate through a nerve pathway, other nerve pathways may also be affected. Accordingly, in certain instances, impairment may also include cognitive impairment or voluntary muscle control of some form.

“subject”—the stimulated and monitored individual of the invention whose degree of impairment is to be determined. In most instances, the subject is human however in certain instances, use of the invention may extend to pets, farm animals, zoo animals or other responsive animals.

“system”—the invention configured to employ a distributed means for execution of tasks utilizing the electronic capabilities of one or more devices in communication with the internet and internet services and coordinating activities and data through communicated software and/or instruction in an adaptable software architecture that may be expandable from one to two or more devices.

“user”—the user is the operator of a device or system employing the method of the invention. In certain instances, the user and subject may be the same individual. The operator of the device might be a law enforcement officer.

### DETAILED DESCRIPTION OF THE INVENTION

The invention described herein presents a method, systems, and devices for determination of impairment through measurement of at least one response to at least one stimulus initiating that response. These are described in greater detail below.

### Invention Overview

The major steps of the method of the invention are:
1. Stimulate a subject to invoke an involuntary muscle movement
2. Record the response to this stimulus both prior to, during, and following the stimulation
3. Analyze the data from this response, e.g., magnitude and timing of response, to arrive at one or metrics indicative of the response
4. Compare this metric against one or more standards, e.g. tables or computational values, to determine the likelihood of impairment.

Additional permutations and variations of the major steps noted above are readily conceivable, e.g., multiple stimuli, etc., and therefore the scope of the invention is not restricted to these steps.

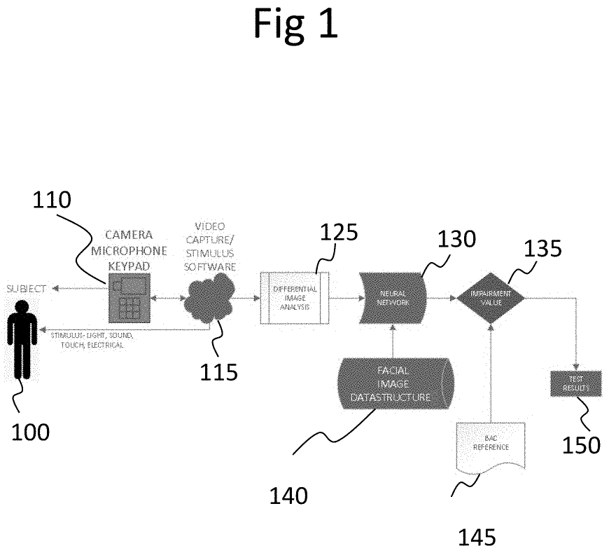

As shown in FIG. 1, an embodiment of a system utilizing the method includes a device 110 that enables initiation of stimulus to subject 100 suitable for resulting in at least one involuntary muscle movement which is recorded by the device. These activities are enabled by video capture/stimulus software 115, preferably located in device 110. In form, device 110 may be a suitable configured smart device, e.g., a smart phone, or alternatively a device constructed wholly or in part to enabling the stimulus, recording of response and possible analysis/transmittal of data and/or analysis.

After completion of the stimulation and response measurement, the data captured by software 115 is then provided to differential image analysis software 125 which may be contained at least in part within device 110 or the data may be transmitted for additional analysis to computational resources located elsewhere. Contained with image analysis software functions are neural net (or machine learning) computational system 130, and facial image data structure database 140 that, when employed with measured subject data, enable determination of possible impairment 135 when compared against equivalent impairment values such as established BAC (blood alcohol content) reference parameters 145 or by other metrics. Upon determination of impairment 135, the results 150 are then presented, e.g., either in device 110 display or elsewhere.

It will be readily understood that significantly greater computational resources are generally available through internet-based activities, e.g., through the cloud, therefore in various embodiments, the device may only provide initial processing of the data and then transmit these processed data to the cloud for further computational and comparative analyses. For example, if image analysis is employed, the raw pixelated images may be analyzed on the device through various software programs and then results from this analysis, e.g., coordinates or dimensions of a body region such as an eye pupil, taking during the process may be transmitted for additional analysis and comparison.

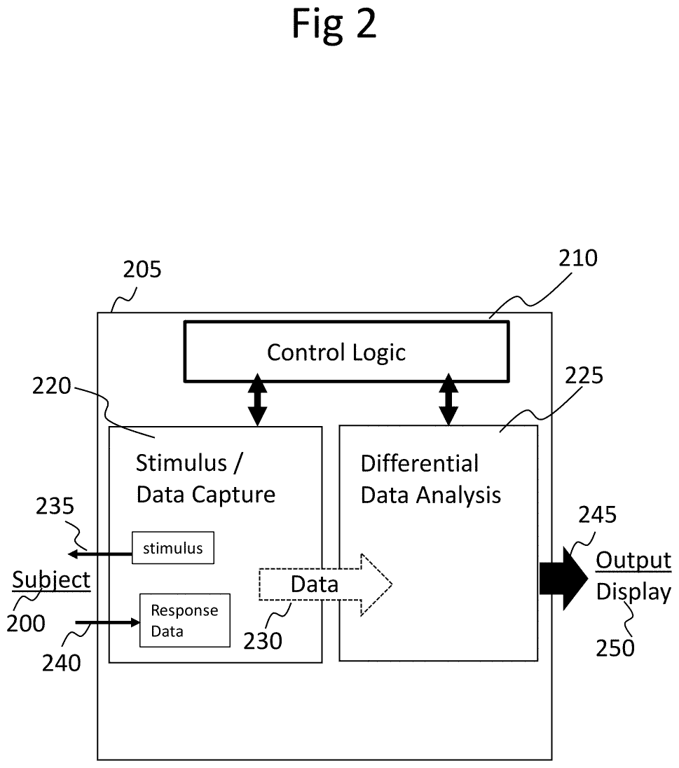

FIG. 2 presents a diagram representative of possible structure software utilized in a device to perform the entirety of the method. It will be readily understood that needed mechanical and electronic components as well as power are readily constructed or obtained by those skilled in the art of electronic manufacture or these features may be contained within existing devices, e.g., smart phones and therefore may just require software enabling the invention. Software 205 contains control logic software 210 that governs the activities of both obtaining response data (including stimulus management) 220 and data analysis software 225. In simplest form, control logic software 210 acts as a state machine initiating the sequential execution commands upon receipt of various inputs, e.g., initiation of activities from keyboard input, activation of data recording indicated by arrow 240 measured from subject 200, including the presentation of stimulus to subject 200 indicated by arrow 235.

In one embodiment, control logic software 210 initiates activities upon receipt of input from device user through keyboard input or other form of input method, such as voice commands utilizing components and software. In one embodiment, once the system is initiated, a signal that the system is ready may be presented to the user by control logic software 210. As part of being initiated and ready for use, functions performed may include identification of device functionalities, thereby enabling automatic tailoring of the software commands to the device. This initiation may also include initial imaging of the subject to ensure appropriate orientation/discernment by software of desired body feature, e.g., the iris and pupil of one eye. The alert signal may be of a variety of forms, e.g., audible beep, audible instructions/commands, visible light or light flash, icon display or test graphic, or tactile stimulation such as a vibration or impulse.

Upon receipt of the readiness signal, the user then may initiate the stimulus 235 by depressing a key, voice command or other means of activation on device 110. The control logic software upon receipt of this input begins to record the subject then initiates the stimulus 235, e.g., causes a flash of light, while also recording one or more aspects of the subject 200 physical status or emotional response, e.g., a full motion video recording of subject's face, or portion of the face such as one or both eyes. In alternate scenarios, the stimulus is initiated automatically, e.g., a few seconds after a warning beep.

In one embodiment, there is a slight delay between initiating of the subject status recording 240 and delivery of stimulus 235 such that the recording starts before the stimulus is delivered, e.g., 0.5 sec prior, to enable one or more measurements to be obtained prior to stimulation. This feature also enables the timing of stimulus delivery to be contained within the overall timing of record data.

When light stimulation is employed, one form of stimulus is that of a bright momentary flash of visible white light encompassing multiple frequencies of light. Alternate embodiments may employ a strobing light with a set number of flashes whose timing and numbers of flashes is preset or responsive to measured parameters. These light bursts are preferably emitted by device of the invention such as device 110 or by a component in direct communication with or affixed to device 110. Autonomic pathways such as those involved with the pupillary light reflex are useful targets for response probing within the present invention as these are involuntary nervous system pathways. Therefore, a measurement target of the present invention may be eye movement includes pupil constriction in response to light stimuli, as these are generally considered to be under autonomic nervous control. In alternate scenarios, additional pupillary response parameters, e.g., random motion prior to stimulation, may be employed for evaluation.

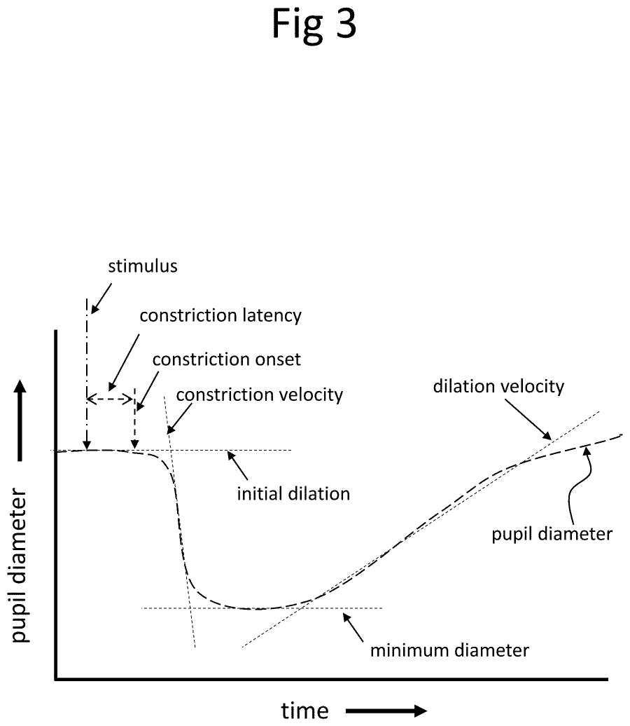

A hypothetical pupil contraction then dilation following application of a brief stimulus e.g., a bright flash of light is presented in FIG. 3. Multiple parameters may be derived from the tracking of the pupil's diameter 370 prior to and following the stimulation 350. Among these are the initial pupil dilation 310, i.e., pupil diameter prior to stimulus application, the latency between the application of the stimulus and the pupil's contraction response 340, the maximal contraction velocity 320, the minimal diameter of the pupil following contraction 380, and the rate of recovery or velocity of dilation of the pupil as the pupil returns to the pre-stimulation state 360. Additional metrics may be further derived from these or other metrics associated with the pupil diameter, e.g., ratios of initial pupil diameter to minimum dilation, terms or equations representative of overall response shape or portions of the response shape, etc. Accordingly, the scope of the invention is not restricted to those parameters described above.

As intoxicants such as alcohol, marijuana or other drugs that result in impairment typically increase involuntary reaction time, e.g., a delay in the constriction onset 330 of the pupil in response to bright light, the present invention has utility for those applications where evaluation of possible intoxicant use or drug use that may result in impairment are desired, e.g., remote telemedicine tracking opioid or other drug use, evaluation of drivers possibly being under the influence of alcohol or marijuana, etc. Other impairments, e.g., chronic pain, may result in other aspects of pupil characteristics, e.g., pupil diameter variance in the absence of stimulation, to be ascertained during the monitoring before, during, and after stimulation.

The intensity of the light delivered as a stimulus and received in the subject's eye is preferably intense enough to elicit a pupil contraction in a subject unaffected by drugs, disease, or alcohol. The intensity supplied may include factors such as the ambient light intensity, the stimulating light source intensity, the distance between the light source and subject's eye and any other factors such as lens, etc. that may impact the photons transmitted from the source to the eye, in particular the retina of the eye. In certain instances, the subject may need to remove any prescription eyewear such as glasses, contact lenses, etc. to enable the light to be delivered with enough intensity and the measurements of pupil response to be accomplished.

The use of a white light enables a broad spectrum of photoreceptors to be stimulated thereby ensuring a response across a variety of health conditions. However, in certain circumstances one or more discrete frequencies of light may be employed, e.g., a blue light or a red light. This may be done for a variety of reasons, e.g., to test responsiveness of the subject to one or more frequencies of light that may be associated with a health condition or the body's ability to sense different frequencies of light, e.g., differential evaluation of retinal cones (red light) versus rods (blue light), or for ease of manufacture and/or cost.

The duration of the light stimulation is preferably short enough to prevent overlap in time between the onset of the light stimulation and the body's response to this stimulation. For example, a typical pupil contraction response measured as the beginning of contraction occurs approximately 200 msec following the onset of light stimulation. Accordingly, in preferred embodiments, the flash duration is on the order of a few milliseconds to enable video recording of the subject prior to response without image quality interfering “noise” from the flash itself. Video recording for imaging purposes are preferably done in one or more visible or infrared wavelengths.

In alternate scenarios, the duration of the flash may be extended, e.g., tens of seconds, to result in a full stimulation of one or more physiological muscle movements whose recovery, post-stimulation, is recorded. In yet other scenarios, a combination of stimuli is applied, e.g., light irradiation for several seconds, followed by a period of recovery then followed by a brief light stimulus, to enable further insight into the physiological status of the subject. Two or more stimulus pulse measurement may be employed, e.g. repetitive sets of stimulus/recovery, to gain additional insight such as depletion of neurotransmitters or other factors involved in signal conduction and muscle activation.

In various embodiments, invention software may also prompt the user to correctly position the recording system, e.g., the camera, after initiating initial image analysis in order to obtain the desired measurements and utilize one or more functions of a device such as device 110, stimulus/data capture software 220 and data analysis software 225 and other software to accomplish this task.

Such positioning may include both region of the body to be measured being appropriately addressed as well as the distance desired for proper measurement. For example, in the instance of measuring facial muscle response including pupil constriction, the system may employ a video-based view superimposing a mathematical construct of the face, e.g., an outline, on the subject's image in a display. By predefined criteria, the face construct can then be determined to be in the correct orientation and distance to enable measurements to be taken. Prompts by the system may be delivered to guide the user into the correct orientation of the device, e.g., through visual instructions on a display or audible commands or tones.

In the instance where the device 110 is a smart phone and the body response to be measured is pupil constriction initiated by a flash from the device, an optimal distance for the phone may be on the order of 10 cm to 50 cm from the subject's face. Other distances may be employed however, e.g., based upon camera resolution, body region to be measured, etc., and therefore the scope of the invention is not bound to any one distance between the subject and device 110.

It is noted that in preferred embodiments the device 110 is positioned away from the target body region and not in direct contact with the subject's skin. In certain other embodiments, a structure, e.g., a resting scaffold, is used. The subject may place their face or body region on this structure to facilitate use of the invention, e.g., ensure a fixed geometry with device use for visual recording, reduction of ambient lighting, or to enable contact between skin and the stimulus source, e.g., an electrode positioned on the skin surface for delivery of a mild shock invoking involuntary muscle movement.

Upon achieving appropriate orientation and distance, the system may then alert the user that the system is ready to deliver the stimulus and take measurements, e.g., with a visual alert or audible tone. In other cases, no alert or warning may be given to ensure maximal surprise on the part of the monitored subject.

The method of the invention is not restricted to the employment of visual light as a stimulus nor the use of a video recording as data acquisition. For example, a small electric shock suitable for providing a sensation to the user may be delivered in certain embodiments. Changes provoked by such a stimulus may be monitored using a photonic capture approach, e.g., a camera or camera function within a device, or alternatively, a measurement such as that employed in electromyography measuring voltage changes may be employed to detect triggering of muscle activity.

In other instances, movement detectors, e.g., accelerometers, may be positioned on the body region or incorporated into the stimulus device, e.g., a smartphone, and utilized for the purpose of detection of muscle movement. In still other instances, other methods, e.g., audio recordings or infrared movement detectors, may be employed to detect motion or other involuntary responses.

In yet other embodiments, an audible tone sufficiently loud to produce a “startle response” in the subject may be employed as a stimulus. Alternatively, some form of physical contact, e.g., a sudden tap to the subject, may be utilized or other stimuli as described herein.

In yet still other embodiments, one or all aspects of the present invention may be contained or associated with devices having an additional function or use. For example, a diabetes blood test which involves a finger prick to draw blood may in certain instances combined with methods of recording involuntary movements, e.g., accelerometers positioned on the skin, to record the reactive response frequently associated with a needle stick. Alternatively, a physiological recording device, e.g., a pulse oximeter, may be configured such that it also performs the tasks inherent in the present invention through incorporation of additional components and software in addition to its primary functions of measuring blood oxygenation and pulse rate.

In short, a variety of techniques or combination of techniques may be employed to deliver one or more stimuli to a subject and a variety of techniques or combinations of techniques may be employed for measuring the involuntary movement of one or more regions of a body and therefore the scope of the present invention is not confined to the examples of these presented herein.

In most instances, the stimulus applied will be sufficient to result in a measurable change in at least one body muscle or activity associated with impairment and that the recording or measurement tool is sensitive to this change and has sufficient resolution to detect the change associated with impairment by either timescale of the monitoring or sensitivity of measurement. In related instances, the stimulus and the lack of response by a body region may in itself represent a degree of impairment when an unimpaired individual would be anticipated to have a response.

In one embodiment, the monitoring of subject response to stimulus for obtaining data is done after a set period of time following activation of the system, e.g., several seconds. The timing of this period may vary upon the nature of stimulus applied and the body response monitored. In most instances, e.g., iris contraction reducing the size of the pupil in response to a light flash and recovery from the flash, this period consists of a short period of time, e.g., a few seconds to several seconds, and therefore the recording period would encompass this anticipated time. In alternate embodiments, the recording period may vary, e.g., dependent upon the degree of measured recovery or some other metric such as available memory or power.

The period of an involuntary response onset is typically less than 1 second after stimulation plus a few more seconds for recovery. This period of time is fortuitously able to be recorded by the memory capacity at a high video frame rate by most advanced smart phones. In other instances, the normal frame rate of recording, e.g., approximately 30 msec per frame, may be employed. Such recordings may have a corresponding increased error of a single calculated parameter dependent on a dynamic response time, e.g., the onset of pupil contraction, due to the decreased number of data points acquired during a set period of time as compared to the short time intervals and greater quantity of data associated with a higher frame rate. In such instances, a variety of mathematical approaches may be used to combine several calculated parameters to achieve a desired level of confidence in the assessment of impairment.

In alternate scenarios, a more extended monitoring time may be employed, e.g., tens of seconds, or minutes, for a variety of purposes, e.g., to enable additional stimuli to be applied, stimulus response recovery times to be tracked, etc. Accordingly, within the scope of the invention, the period of monitoring is not limited to one set period of time.

In one embodiment, the data set comprising recorded subject response(s) including time interval data, e.g., time stamps on each image file or frames of video taken at a known rate plus an initiation time, are provided to differential image analysis 225 as instructed by command of control logic 210, e.g., transference of electronic data from stored memory buffer to one or more analysis routines associated with image analysis.

In alternate scenarios, data may be sent to analysis effectively as acquired or in batches, e.g., to accommodate video memory buffer capacity. In yet other scenarios, the data are stored for a period of time prior to analysis. Such storage may enable additional inputs of data, e.g., subjective scores of overall body control, to be included in the subsequent analysis.

Upon completion of the monitoring, stimulus data capture software 220 alerts control logic software 210, which in turn instructs transmission or ability to access data to differential data analysis software 230. In some embodiments, data capture software 220 is located in the same device as control logic software 210 and analysis software 230. In alternate scenarios, these functionalities may be distributed between two or more devices or electronic systems, e.g., transmission of data, processed data or initial analysis of data, to the internet, i.e., to the “cloud”, as part of a system of the invention. Accordingly, the physical location of these storages and analysis functions is not constrained to any one device, electronic system or location thereby facilitating use of the invention in applications such as those occurring in telemedicine or for remote data review by managing authorities.

Upon accessing measured data 230, differential image analysis software 225 then processes the data, upon receipt of command to do so from control logic 210. This processing may take the form of determining response time to stimulus and then as a second function, determines whether this response time is within normal ranges for the subject or possibly more rapid or slow than the reference data set utilized for analysis would predict.

Analysis may be performed with additional data input, e.g., population data, subject age, gender, health status, medications, etc. to more precisely define normative parameter range(s) anticipated for the subject and facilitate with greater accuracy the likelihood of deviation from that range, i.e., impairment. This additional data may be entered into device 110 by user in response to one or more display queries, or may come from other sources, e.g., electronic health records, Dept. of Motor Vehicles records, etc., and accessed through prior storage or an application programming interface (API) links in the “cloud” to the system of the invention. The scope of the invention is not limited by the type or origin of any additional data utilized for the purpose of analysis.

A preferred early step in the image analysis 225 for those forms of the invention employing video data sets is that of frame-by-frame image analysis of the data to plot points on a 2-dimensional or 3-dimensional mathematical grid (geometry) representative of one or more body features, e.g., points representative of the pupil located within the iris transposed to grid coordinates. Removal of bad data such as images exhibiting out of range parameters, e.g., parameters that cannot be physically real, may also be performed at this time.

To accomplish this task, a variety of software tools developed for facial biometric and facial recognition purposes are readily available, e.g., ones that create such “grid-like” transposed data sets and may be incorporated into differential image analysis 225. Such software may be also employed to aid in verification of the identity of the measured subject. For example, during a measurement recording sequence (or during the initiation phase), one or more images of a subject's physiognomy, e.g., face structure or iris structure, may be analyzed and compared to a database to ensure that the measured values arise from the assumed individual. Such forms of identification employing one or more images or data sets from the measurement process may be automatically included into the measurement routine. Such automatic incorporation of identification may be beneficial where the method and devices of the invention are employed in a remote measurement scenario, e.g., by a clinician in telecommunication with a patient who employs a smartphone for conducting the measurement process to ensure that the data arises from the patient.

A preferred source of facial (or portions thereof) biometry software is open-source code software constructed for this purpose and readily adapted to the needs of this invention. Open-source code exists that tracks the human face, resolves the geometry and location of the facial musculature, and can track changes in these geometries within a data set. Open-source code has many advantages including crowd error correction, testing and documentation. Open-source code is freely available software code released into the public domain by the author/programmer or an organization. Examples of such open-source software include OpenCV (computer vision), TensorFlow, as well as other various private and public repositories of software.

Once a set of coordinate points has been determined and measured throughout the desired time period(s), a variety of analytic approaches for determining change associated with movement may then be applied to the data set to determine onset of movement and the timing of this onset and recovery relative to stimulus delivery.

One such approach is that of multivariate analysis such as principal component analysis, to identify changing parameters within the data set. In yet other forms, derivatives of these values, including the relative magnitude of the values, derivatives of these values such as those yielding rate of change or acceleration/deceleration maxima may be employed.

Alternatively, techniques such as neural net or machine learning approaches may be employed. In such instances, the learning sets may be within in the individual data sets themselves with the system trained to identify the timing or magnitude of significant change or rate of these changes from baseline, i.e., utilizing data from non-stimulated responses that occur before stimulus reaction occurs and evaluating significant threshold change from these baseline values.

Returning to FIG. 3, and the eye pupil in particular, the pupillary response of constriction is primarily controlled by a form of innervation, parasympathetic, that differs as compared to the primarily sympathetic innervation mediating dilation. This difference in innervation potentially yields different reaction responses to light stimulation for various sections of the overall response. That is, each region of the overall response curve depicted may reflect a separate physiological state or condition associated with the respective form of innervation, i.e., each segment may reflect different underlying physiological phenomena. Accordingly, in preferred forms of the invention, analysis such as that may be performed through machine learning enables determination and comparison of individual sections of the response and enable the discrimination between normative and abnormal ranges for one or more of these regions. Such forms of analysis may enable creation of a “discriminator” within the overall analytical process to identify the portion of the response at variance with normative values to that portion, and possibly the underlying physiological basis for this variance. This approach differs from those forms of analysis which consider the overall response as a whole and thereby lessen sensitivity to the identification of any one portion of the response curve and its associated underlying physiology as being abnormal.

Forms of machine learning may also enable employment of the invention in multiple environments or environmental conditions, such as external temperature, time of day, or lighting, where such information is supplied to the system, e.g., data measured by one or more sensors, calculated, or directly inputted from a user. For example, consider the pupil constriction/dilation response shown in FIG. 3. Ambient light tends to affect the curve by vertical displacement of the initial pupil diameter and proportionally displace the values of the subsequent pupil diameter curve. However, in certain instances, e.g., with excessive opioid use, the initial pupil diameter may be less than predicted by the normative values for local ambient lighting conditions, i.e., a condition of miosis. Accordingly, the system learns what normative responses should be expected for various ambient lighting conditions, and as a result, the system may operate without needing to control the ambient light through use of a darkened room or through a structure shielding the eye from outside, ambient light, as compared to traditional pupillometers. Upon receipt of environmental information, e.g., from one or more sensors regarding ambient lighting, computational activities of the invention may then determine whether the pupil response measured is indicative of being within a normative range or indicative of impairment for the local environment experienced.

As refinements of mathematical approaches, methods for noise reduction in the data set, e.g., rejection of outlying data arising from spurious signals or unwanted subject movement, or averaging of parameter values over several time intervals to smooth noise, may be employed to facilitate analysis. In yet other instances, biometric points not associated with muscle movement, e.g., the tip of the nose, may be used as referential (fiducial) points to aid in normalizing data values, e.g., magnitudes, from frame to frame in video data sets, e.g., employ video stabilization techniques, and thereby facilitate analysis. Various aspects of these issues, such as noise reduction and use of fiducial markers, are well known to those skilled in image or signal analysis. In many instances, these issues may be directly addressed through suitable forms of open-source software. However, certain forms of data artifacts, such as an eye blink reflex during measurement, may disrupt the data to such an extent that an error message may occur and be transmitted to the user, with the intent that the measurement process needs to be repeated to acquire useful data.

It is understood that other measurement techniques, e.g., accelerometers affixed to the individual, may yield data of a different form than that of video (pixel-based) approaches. However, the tools for analyzing forms of data strings that incorporate a temporal and scalar component (magnitude of response) for change from baseline conditions as well as noise mitigation are well known to those skilled in the art of data analysis and therefore other measurement approaches are included within the present invention and the methods of analysis employed.

In short, a variety of mathematical tools and software exist for the purpose of analysis and are well known to those skilled in the art of data analysis. Accordingly, the scope of the invention is not restricted to any one technique or method for determining the onset, magnitude, or any other related parameter(s) of the stimulus response within a data set.

Determination of how the timing or magnitude of the response of the data values associated with the response compares to normative or impaired values is utilized in various embodiments of the invention to assess the likelihood and degree of possible impairment. One means of accomplishing this objective is the mathematical comparison between the observed response value(s) of one or more parameters or sections of a response versus those same response parameters in comparative data sets that preferably includes both normative and impaired values. In certain instances, mathematical transforms of the data sets, e.g., parameters, equations, or algorithms defining the data sets or values and the variance associated with these may be employed.

It is noted that the origin of the comparative data sets may come from a variety of sources. For example, measurement using the tools of the present invention in normal or non-impaired individuals as well as measurement of individuals deemed to be impaired using other metrics or standards, e.g., blood alcohol content measurement, may be readily employed to construct these data sets. Accordingly, the scope of the invention is not limited to any one approach or method for construction of comparative data sets.

In simple forms of these analyses, standard statistical tests, e.g., Student's T Test or ANOVA, linear regression, or Gaussian Processes, may be employed to determine the likelihood of difference between values or groups of values. In other instances, a multitude of data values may be reduced in complexity or dimensionality to facilitate analysis. Such approaches such as Principal Components Analysis, Dimensional Reduction, Multidimensional Scaling, Factor Analysis, or similar approaches are well known to those skilled in the art of data analysis and may be employed to facilitate analyses.

In various instances, additional data may be employed to maximize the comparative nature of the assessment, e.g., employing other information such as age, height, weight, gender, ethnicity, medicines used, or health status that more closely relate to the measured subject in the selection of comparative data sets or algorithms to be employed.

In alternate embodiments, only one comparative data set or representative set of parameters or algorithms are utilized to determine likelihood that the observed data value(s) fall within that of the comparative data set or range. For example, a mathematical model approach may employ training against multiple individual datasets so observed impairment determinants arising from this modeling may then be matched up against an individual's experienced dataset for comparative evaluation. These datasets may arise from the measured subject, being taken at least one earlier point in time when the subject's physiological status was known then trended or modeled longitudinally over time. Conversely, the modeling may be from datasets arising from a combination of individuals which may or may not include the subject themselves.

In still other embodiments, the comparative data sets do not identically match that of the response data. In such instances, e.g., where age range of comparative data sets do not encompass those of the subject, an extrapolation based upon one or more parameters that relate to the out-of-range value may be employed to adjust the comparative data set or parameter for the analysis. Conversely, the subject data values may likewise be adjusted to provide an improvement in the analysis. Quantitative scaling, e.g., not necessarily linear extrapolations, may be employed for these purposes, as well.

In yet other embodiments, the comparative data sets or values representative of these sets are abstracted from data arising from different measured values, e.g., different body sites or measurement techniques, or theoretical considerations. In such instances, scaling or adjustment factors may be employed to enable comparison. For example, if the measured value derives from pupil constriction onset and the comparative data sets is the patella reflex, a timing constant may be employed to compensate for the different neural pathways utilized in the autonomic responses. That is, impairment due to intoxicants may affect the entire human neurological system but in different magnitudes of exhibited reflex responses.

In still other embodiments, subjective data, e.g., examiner subjective scoring input from a test such as the SFST test, may be incorporated in the analysis of impairment. Additional metrics, e.g., time of day, subject medical history or disease conditions, may also be incorporated in the analytical process determining likelihood and extent of impairment.

Alternatively, more sophisticated approaches may be used such as supervised machine learning utilizing decision trees, neural networks, support vector machines, or similar approaches well known to those skilled in the art of data analysis may be employed to determine likelihood and degree of impairment.

In yet still other embodiments, use may be made of emotional recognition software in conjunction with impaired autonomic response determinations to define the degree of impairment more accurately. There are multiple emotions whose interaction might have diagnostic value. The subject might show ‘disgust’ at being stopped for evaluation then show ‘surprise’ at the stimulus then show ‘sad’ after the test is done. ‘Confused’ readings might indicate inability to cope with the situation, possibly indicating a depressive state, possible anxiety, or psychoactive state (possibly of a drug) affecting the mind. Illicit drugs might affect the human brain in this manner. Techniques such as machine learning of associations/interactions across facial datasets with multiple dimensions may be employed to enable various embodiments of the present invention and therefore are included in the scope of the present invention. One means for enabling determination of emotional status may be to include an image analysis approach such as the Facial Action Coding System (FACS). FACS refers to a set of facial muscle movements that correspond to a displayed emotion. Using FACS, one is able to determine the displayed emotion of a participant, within a certain likelihood. Originally created by Carl-Herman Hjortsjö with 23 facial motion units, it was subsequently developed further by Paul Ekman, and Wallace Friesen and is adaptable to applications such as this invention.

Given the possible forms and varieties of mathematical approaches for evaluation of impairment, the scope of the invention is correspondingly not constrained to any one type or method of mathematical analysis.

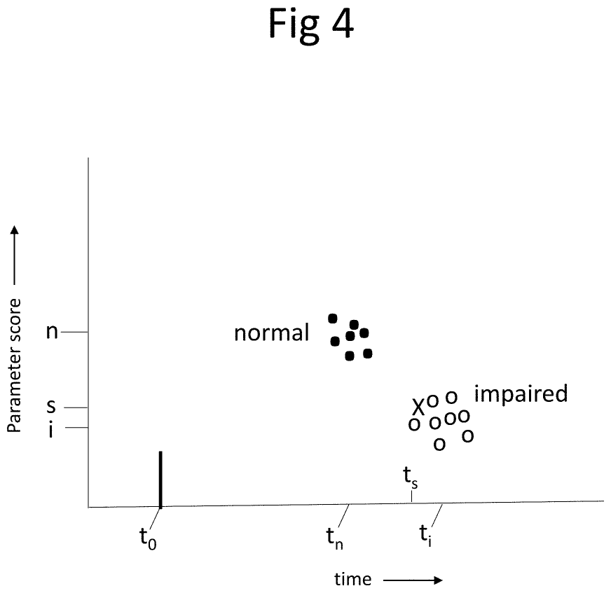

An example of a simple form of data analysis is presented in FIG. 4. FIG. 4 shows the discrimination of values grouped with a classifier algorithm. This hypothetical graph indicates a subject's data value (X) plotted against two comparative data sets, normal (●) and impaired (◯). In this hypothetical example, the parameter score may be the rate of pupil contraction following a flash of light. As shown, the mean of normal data set time after stimulus (t₀) is at t=tₙ and has a parameter value of n. The mean of impaired data set time after stimulus is tᵢ and has a parameter value of i. The hypothetical subject's time is indicated by tₛ and has a parameter value of s.

It is a common statistical exercise to test if parameter X's time or parameter score is more likely to be part of the normal population or of the impaired population. Using basic forms of statistical analyses, the mean and standard deviations of both comparative data sets may be readily computed and the probability that X is more likely associated with either of the two groups may be readily determined. This is a common statistical exercise, e.g., done using classical statistical techniques, or by using a machine learning based classifier algorithm. More complex forms of analysis may be employed and are well known to those skilled in the art of statistics or probability analysis.

Having determined the likelihood and extent of impairment, additional analysis may include whether such impairment exceeds a pre-determined level or threshold. The scoring may be directly associated with a threshold value or indirectly as a probability. Impairment scores associated with a parameter score such as pupil constriction time may be then correlated to an existing standard such as blood alcohol content.

In such instances, the blood alcohol content cut-off maxima are well established, e.g., 0.08% in most states, and if the eye constriction response comparative data set has been previously correlated to blood alcohol content, then by inference, a certain score of pupil constriction onset delay may be assumed to correspond to a certain impairment level or Blood Alcohol Content (BAC) equivalent.

In a similar fashion, determination of impairment levels through use of one or more metrics, e.g., pupil constriction, may result in a universal standard of impairment separate from the causal agent of impairment. For example, if an individual who has consumed alcohol scores a certain impairment value during screening, a second individual who scores the same value may be assumed to have the same degree of impairment even though alcohol is not the causal agent of impairment. For example, an individual may be judged to be impaired if the pupil constriction reaction time is increased by more than 125 msec.

Upon determination of likelihood and extent of impairment, the differential analysis 225 informs control logic 210. Control logic 210 in preferred embodiments then directs results to output 245. In preferred embodiments output 245 is a visual display located on device 110. The output may present numeric results of the analysis as well as possibly additional information, e.g., severity of the impairment, etc.

In addition to visual display of numeric values, the output may result in forms of alert, e.g., flashing lights, vibration or audible tones, indicative of subject's impairment status or pass/fail status. Fail indication could be passed on to the operating vehicles control system to engage a ‘safe mode’ whereby the vehicle performs a self-park maneuver and alerts a monitoring system of the vehicle's status/driver's condition.

In yet other embodiments, the response value may prompt one or more responses. These responses may include, but are not limited to, cause an automatic action by another operational system that is in contact with the output, cause additional information to be presented by device 110 or present queries relevant to the stage of impairment. Questions may include questions relative to health status, e.g., recent drug or alcohol use, or may provide verbal or visual suggestions to additional services or activities that may be of use to the subject, e.g., web-based links to ride share services, hotel services, food services, or suggested rehabilitation service providers.

In still other embodiments, the results of the analysis possibly with measured data and other data associated with the screening is stored or transmitted. Storage permits subsequent retrieval and use, e.g., trend analysis, third party review, etc., and transmission may enable secondary storage, e.g., in the internet cloud, and additional review by others or possible follow-on automated actions or activities resultant from the screening.

### Devices of the Invention

In one embodiment of the invention, the method of the invention is enabled in a single self-contained device. Such a device is a computational device having sufficient measurement capabilities such as visual frame recording rate and resolution plus analytical processing function, memory, display, and power to accomplish the desired functions of the method.

In alternate embodiments, a system having at least one device and the internet “cloud” capabilities are employed. For example, the initial activities of sample collection may be accomplished using a device, e.g., application of stimulus and analysis yielding one or more parameters associated with the physiological response, e.g., latency or duration of pupil dilation. Results of such analysis may then be transmitted to a second, internet-associated, analytical process, e.g., transmitted to the “cloud”, for comparative assessment of these parameters to population-based responses or further for further computation. System-based enablement of the invention affords the advantageous properties associated with internet such as scale-ability—the ability to handle data from a multitude of devices, and port-ability—the ability access and utilize the invention or data arising from the invention in a variety of locations and use scenarios.

For measurements employing video recording of a stimulus such as a flash of light or brief loud sound, a “smart” phone may serve as a preferred device of the invention whether as a stand-alone device or as a component of the system of the invention. As video frame rate on modern smart phones in slow motion configuration is on the order of 120 frames per second, and super slow motion has frame rates on the order of 400 frames per second, these devices have sufficient data capture speed to discriminate events with resolution of 10 msec or less. Such resolution enables collection of data allowing discriminate between a normal pupil constriction time onset following flash stimulation on the order of 200 msec and that of an impaired constriction onset which may be 300 msec or more. In other instances, the normal frame rate of the “smart” phone, e.g., approximately 30 frames per second, may suffice to provide useful data for determination of impairment, especially if analyzed using multiple parameters acquired in various phases of the measurement process.

A further advantage of employing a suitably configured smart phone is that, in certain instances, the user of the system can also be the measured subject. That is, with ability to record from either face of the phone, i.e., the front having a display and the back, as well as the ability to deliver a light stimulus, i.e., from the front display or back facing camera flash feature, a user may hold the phone in such a manner to both enter commands through the touch pad display, and in so-called “selfie” mode, hold the camera to enable the stimulus to be delivered and a video record obtained. In other applications, the back side of the camera and flash are employed on a subject while the user initiates commands with display on the from or using audible control.

Impairment evaluation software to be employed includes the use of Tensorflow/JavaScript programs which take advantages of installed web-browser window software native to the majority of phone systems, e.g., Internet explorer, Chrome, Firefox, Safari, Edge, etc. Other software may be present native to the phone as preinstalled software, e.g., video capture, or readily downloaded as an app or script to be run, e.g., control software. As such, one can readily envisage the ability to download an app into a “smart” phone that enables at least a portion of the method of the invention to be performed by the user of the phone. These approaches would employ several of the functionalities already present in a phone, e.g., display, flash, video recording, etc.

In other embodiments, dedicated devices having the requisite optical, mechanical, electrical and software functionalities may be purposely constructed for conducting various forms of the present invention. In form, these may be constructed to contain all functions of the invention or only portions of the invention, e.g., stimulation and recording of data, with other non-dedicated devices, e.g., laptop computers, employed to conduct other functions of the invention.

In still other embodiments, one or more requisite functions, e.g., stimulation and recording, may be incorporated into fixed structures or devices having additional uses. For example, the stimulation light source and video recording capability may be built into a control panel or console such that the user of the control panel may be tested for impairment during use, e.g., by a remote third party upon command or by instruction from a computer program, such as remote testing for an employee drug-free environment.

In related embodiments, the device of the invention may be contained in a stand-alone structure, e.g., a kiosk or tabletop unit, that enables an individual to initiate the screening for themselves or for other individuals. In yet other embodiments, more than one subject may be evaluated through use of area-based stimulation, e.g., bright light flash, and suitably configured camera systems. For example, crowd surveillance in a confined space such as a bar, school, or subway station may employ cameras of suitable resolution to enable record facial details from multiple individuals at a single time and thereby enable discernment of possibly impaired, e.g., drugged, individuals within a larger population. As one variation of this embodiment, an entryway video system might identify and evaluate oncoming personnel for emotional state via emotional rendering and notify authorities of at-risk situations.

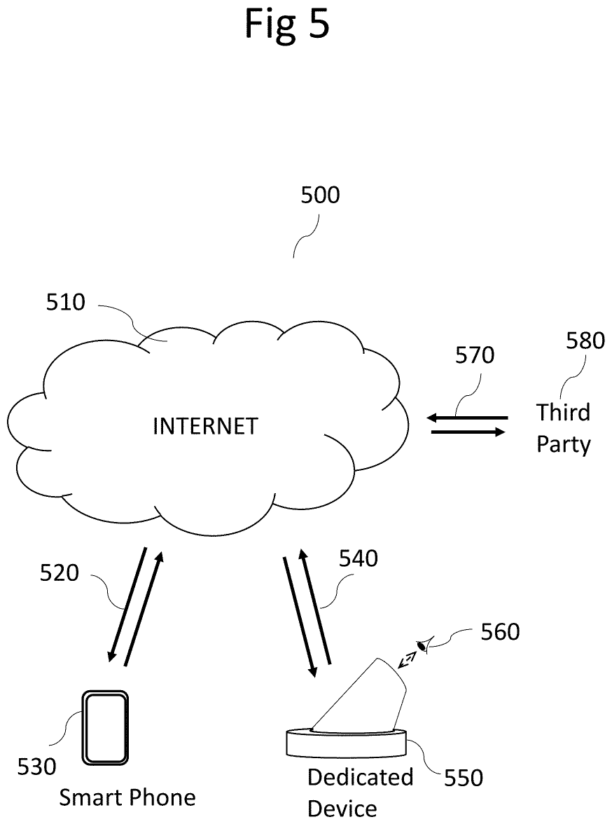

An illustration of how the system of the invention may be employed is shown in FIG. 5. The system 500 employs both the internet “cloud” 510 and two forms of devices, a “smart” phone 530 and a dedicated device 550. The user of smart phone 530 may communicate with system cloud 510 by opening up a web-browser window that in turn enables code such as hypertext markup language (HTML) code that may have components of TensorFlow, Javascript, and other custom code such as APIs, to be employed to communicate with the user, in contrast to the user having to go to an APP store and downloading then opening a stand-alone program (APP) configured for their form of smart device. In greater detail, the browser renders the code, e.g., HTML code, it loads from the URL site, i.e. from the Internet “cloud”, to accomplish the invention. This is different from software code that is downloaded from the APP store and runs in the mobile device.

Two-way communication between device 530 and internet “cloud” 510 is indicated by arrows 520. Other features within browser functionalities ported from internet “cloud” 510 are employed to control and activate the measurement process, including use of the flash, etc. At the completion of the stimulus and recording process, a partial analysis of the image data may be performed to reduce the size of the data packet to be transmitted back to internet “cloud” and its associated computational and storage services, such as those provided by Amazon Web Services, Google Cloud, etc. This reduction would entail programs and mathematical routines ported to the smart phone as a part of the overall use of the system.

The system would also enable use of additional devices effectively simultaneously, as shown by the bi-directional communication 540 between system “cloud” 510 and device 550. In this instance, device 550 may be constructed solely for the purpose of conducting impairment testing, e.g., through the use of light flashing from device 550 into the eye 560 of a subject. Needed electronic circuitry, mechanical switches, display, lights, camera, memory, power, etc. would be present within device 550, with additional impairment assessment computation of acquired data provided by “cloud” 510. In this scenario, a user who is also the subject may activate the system by activating device 550 then upon receiving results from “cloud” 510 may then chose to act upon these results, e.g., forego an activity requiring sobriety.

Also shown in communication with “cloud” 510 is third party 580. This feature of system 500, enabled the directed testing of a subject, e.g., upon instruction from a clinician, an electronic message alerting a user of smart phone 530 may be asked to connect to system 500 and conduct the evaluation. Upon completion of the evaluation, the clinician (third party 580) may receive the findings and incorporate these into the subject's medical history or these may automatically populate into an electronic health record. In alternate scenarios, third party 530 may be a service provider, such as a Lyft or UBER ride service. Upon receipt of impairment information from device 550, a message may then be transmitted back to the user of device 550 through device 550 inquiring if they would like a car driver sent to their location. Additional use scenarios involving various devices, users and third parties are readily conceivable and the scope of the invention is not limited to the examples presented here.

In other embodiments, a component configured to enable one or more functions of the present invention may be affixed or connected to a multifunctional device such as a smart phone or laptop. For example, a structure having a light source, photosensor, optical lens, and a housing intended to reduce ambient light delivery to an eye region when positioned on the face of the individual may be connected to a smart phone through a USB cable or similar means in order to accomplish one or more of the tasks of the present invention.

In still other embodiments, a separate device such as a patch may be affixed to a subject, possibly in wireless connection to control logic, wherein the patch is configured to deliver a stimulus, e.g., vibration, electrical shock, on command and the response recorded by a separate device. In certain instances, the patch may also record the response, e.g., muscle twitch, etc. such as by using accelerometers or voltage sensors. In further embodiments, such patches may also include medications or drugs, e.g., stimulants, that may be delivered by a structure on the patch in response to determination of the degree of impairment.

In yet other embodiments, the method of the invention may be contained within a form of telecommunications, e.g., on-line meeting systems such as Zoom meetings, whereby the user at one location may instruct the subject to initiate a stimulus, e.g., a light flash, which will be monitored using the on-line meeting video functions and analyzed for impairment using internet-based analytical tools.

It will be recognized that various combinations or alternate forms of the steps of the method, system, and devices presented herein are readily conceivable and therefore, the scope of the invention is not limited to those forms presented herein.

### EXAMPLES OF USE

Illustrations of how the process of the present invention may be used are presented in the following examples:

### Example A). Impaired Vehicle Operation

For determination of whether an operator of a vehicle, e.g., car, truck, boat, etc., is impaired, one use would be for authority figure such as a police officer or supervisor utilizing the method and a device to screen the operator, i.e., subject. In such instances, utilization of a suitably configured smart phone may prove a practical means for rapid evaluation and provide a more quantitative assessment than a test such as the subjectively scored SF ST.

In other instances, the screening capability may be incorporated into an instrument panel or as a stand-alone device that is able to govern operation of the vehicle. In these scenarios, the operator themselves may initiate the screening process. Upon receiving a score that indicates possible impairment, ability to operate the vehicle would be automatically prohibited. In such scenarios, a “fail” indication could be passed on to the operating vehicle's control system to engage a ‘safe mode’ whereby the vehicle performs a self-park maneuver and alerts a monitoring system of the vehicle's status/driver's condition. As further variation, the screening process may also include some form of identification verification to prevent spoofing or misuse of the system.

### Example B). Self-Assessment in Social Situations

In order to avoid potentially operating a vehicle after consuming alcohol or drugs, a user may employ a smart phone-based system of the invention to self-check on their degree of impairment, i.e., the user is the subject. In certain scenarios, if the impairment registers as significant or possibly significant, an alert may be presented to the user of their status and in further variations, the system may also automatically engage a lockout device to prevent operation of the subject's vehicle or offer a link to a ride sharing service such as LIBER or Lyft to aid the user in avoidance of independently operating a vehicle while in a questionable impairment state.

In a different embodiment of this scenario, a bar or restaurant may have kiosks or devices that contain at least a portion of the system of the invention and thereby enable both service personnel and system users to become aware of their status and thereby avoid escalation of consumption and possibly engage an outside service for transportation, etc.

### Example C). Allowance of Clientele Participation

An establishment where the patrons may actively participate in an activity such as gambling may result in liability to the establishment if the patrons suggest that their impairment resulted in poor performance or financial loss and that the establishment was negligent in assessing their degree of impairment and thereby allowed them to participate when they should not have.

Accordingly, systems of the invention configured to evaluate one or many individuals, perhaps simultaneously, may be employed to ascertain possible impairment of the individual and enable recording of this assessment for future use. In such scenarios, the system may be in the form of devices, e.g., phones or dedicated units, utilized by employees on possibly impaired individuals. In other scenarios, use may be made of existing video monitoring/surveillance capabilities and a stimulus such a bright flash presented, enabling assessment of one or more individuals for impairment effectively simultaneously.

### Example D). Health Status Assessment

Impairment of involuntary reactions may be employed to track various medical conditions and states. Such conditions may include, but are not limited to, whether an individual is taking prescribed medications, such as for pain relief, anti-depressants, sleep aids, etc. which may impair them or in certain disease conditions, e.g., acute conditions such as ischemic stroke or chronic conditions such as type II diabetes mellitus, may also impair their reactions and signal some form of nerve or neurological damage where the present invention may aid in the diagnosis and tracking of progression of the disease. In these instances, the examination by a clinician may be accomplished in person through direct evaluation of the patient or the examination may be accomplished remotely, through the patient self-administering at least a portion of the evaluation, e.g., operating a suitably equipped smart phone upon advice of the clinician, and then transmitting the results to the clinician through the cellular phone network, i.e., as a form of telemedicine.

Ability to download the appropriate code to a suitable device such as a smart phone by a subject or user may be given by a prescription from an attending physician, e.g., where the internet “cloud” employs a means such as a verifiable transmission from the patient compared to a physician enabled permission to enable the communication needed. This prescription may be for certain number of measurement sessions such that the attending clinician may track the individual's status over time, or the prescription may be for more limited use, e.g., a single measurement, where the need for an immediate evaluation may be indicated.

Various forms of the invention may be employed in this use case example. For instance, a hand-held dedicated device may be employed by a first responder, clinician, or other skilled individual to assess degree and possible extent of impairment. In alternate scenarios, a patient may themselves be the user and subject where they employ the system of the invention within a convenient form such as a smart phone to track and help manage disease progression and possible medication usage or for the assessment of an unknown injury or medical condition. medication & treatment efficacy with automated follow-up and condition status tracking

Alternative uses and forms of the systems employed for these uses are readily conceivable and therefore the applications and devices of the present invention are not limited to the examples presented herein.

What is claimed is:

1) A method for the determination of impairment comprised of:
a) Delivery of at least one stimulus to an individual for the purpose of causing an involuntary body response;
b) At least one means of recording at least one involuntary body response before, during and after the applied stimulation to determine the timing of the response to the stimulation;
c) Analysis of the observed timing of the response to a larger dataset enabling determining the likelihood of impairment; and
d) Presentation of the results of the analysis by means of a display or other form of alert.

2) The method of claim 1 also including analysis of the relative timing and magnitude of the measured response.

3) The method of claim 1 where the body response is that of at least one muscle is located on the face of the individual.

4) The method of claim 3 where the muscle in the face includes muscles associated with the iris of the eye.

5) The method of claim 1 also including the use of a least a portion of the recorded data for the purposes of identification of the individual.

6) The method of claim 1 where the stimulus is at least one flash of light.

7) The method of claim 1 where the presentation of results may include: an automatic alert to a third party; an automatic notification to a ride share organization; automatically populate into one or more electronic health records; or automatically result in an inability to operate a vehicle.

8) The method of claim 1 where the analysis also includes one or more additional inputs regarding the ambient environment, such as ambient lighting, ambient temperature, or the time of day.

9) The method of claim 1 where the analysis also includes the use of emotional recognition data in conjunction with the measured involuntary response data.

10) A device for determination of impairment comprised of:
a) At least one component and software capable of delivering at least one stimulus to an individual for the purpose of eliciting at least one involuntary muscle movement;
b) At least one component and software for measuring at least one involuntary muscle response in a stimulated individual;
c) At least one computational component and software for the calculation of a least one parameter enabling the determination of the likelihood of impairment; and
d) At least one display or notification structure and software conveying information to a user regarding at least a portion of the computational results or indicating degree of impairment.

11) The device of claim 10 which is a smart phone or other mobile internet enabled computational device.

12) The device of claim 10 incorporated into a structure having one or more additional functions or uses.

13) The device of claim 10 where the component for stimulus delivery, the component for measurement, or the computational component is a separate structure from at least one of the other components.

14) The device of claim 10 where the stimulus is a flash, synchronized flash, or strobe of light.

15) The device of claim 10 where the measurement is a video recording.

16) A system for the determination of impairment comprised of:
a) A least one device employed for the purpose of delivery of at least one stimulus and the recording of at least one involuntary muscle movement in response to the stimulus;
b) An analysis conducted on at least one of the devices using recorded data from a stimulated involuntary muscle movement;
c) Transmittal of the analysis results and/or data to at least one internet-associated component located elsewhere for further analysis; and
d) The presentation of impairment analysis results to one or more locations from the internet associated component.

17) The system of claim 16 where the location of the presentation of impairment analysis results is at least one of the devices associated with the application of the stimulus or recording of the response of the stimulus.

18) The system of claim 16 configured to interact with a plurality of devices.

19) The system of claim 16 where the device interacts with the Internet-associated component through one or more internet web interfaces or browsers.

20) The system of claim 16 where the system also communicates either instructions or a portion of the analysis or results with at least one other third party through the Internet associated component.

21) The device of claim 16 where the device is a smart phone or other mobile computational device.
